(19) United States
(12) Patent Application Publication
(10) Pub. No.: US 2005/0015101 A1
(43) Pub. Date: Jan. 20, 2005

(54) Leverage locking reversible cyclic suturing and knot-tying device

(76) Inventors: George H. Gibbens III, Shreveport, LA (US); Susan Duke Gibbens, Shreveport, LA (US)

Correspondence Address: RALPH D CHABOT, 2310 E PONDEROSA DR, SUITE 4, CAMARILLO, CA, 93010, US

(21) Appl. No.: 10/812,324

(22) Filed: Mar. 28, 2004

Related U.S. Application Data
(63) Continuation-in-part of application No. 10/263,902, filed on Oct. 3, 2002.
(60) Provisional application No. 60/458,721, filed on Mar. 28, 2003.
(60) Provisional application No. 60/327,704, filed on Oct. 4, 2001.

(51) Int. Cl. A61B 17/04

(52) U.S. Cl. 606/144

(57) ABSTRACT

Embodiments of a suturing device and methods of suturing are disclosed. The device comprises an arcuate needle, a plurality of one way gate mechanisms radially spaced around a portion of the arcuate needle, where each one way gate mechanism engages the needle in one direction, and a driving means for driving the plurality of one way gate mechanism around a portion of the needle such that the needle is incremented along a rotational direction.

### CROSS REFERENCE TO RELATED APPLICATIONS

This application claims the benefit of the filing date of U.S. provisional patent application Ser. No. 60/458,721, filed on Mar. 28, 2003, the disclosure of which is incorporated herein by reference. This application is also a continuation-in-part of patent application Ser. No. 10/263,902, entitled “Cycling suturing and knot-tying device” filed on Oct. 3, 2002, which claims the benefit of U.S. Provisional application Ser. No. 60/327,704, filed Oct. 4, 2001, of which all of the disclosures are hereby incorporated by reference.

### TECHNICAL FIELD

This field of invention relates to suturing devices, specifically to such devices as are used for surgically joining tissue.

### BACKGROUND INFORMATION

Surgeons, doctors and veterinarians commonly treat patients in need of having wound or surgical incision closure performed by using hand-held needle and thread sutures or metal staples. Such suturing methods are also used for procedures that involve joining or connecting without incision. Surgical suturing, using flexible thread comprised of either animal gut or synthetic filaments, is currently limited to the age-old sewing technique of passing a free needle joined to such a filament through a piece of tissue. The needle is held either by hand or by a hand-held clamp while the needle is pressed through the tissue. Once its tip has emerged from the tissue, the rear of the needle is released and the emerging tip is grasped either by hand or by clamp to pull it through the hole the needle has created. The filament is passed through the hole behind the needle. Some of the filament is left protruding from the entrance and some protruding from the exit of said hole. This process is then repeated on a second piece of tissue, which is to be joined to the first. Then the two ends of the filament protrude from the entry hole of the first piece of tissue and the exit hole of the second piece. The filament ends must next be drawn together which pulls the two separate pieces of tissue together. At this point a knot must be tied by use of the fingers or by clamps requiring several passes of the thread. The ends are looped and tightened around each other for each separate knot. The number of knots tied determines how long they will remain joined after the surgery. Some surgeons estimate that one knot may last less than a week in certain applications.

This stitching and tying process is extremely time-consuming, occupying from 25% to 75% of the duration of a major operation. The lengthy process requires the patient to remain under anesthesia for extended periods of time, thereby increasing the risk of complications not only from the anesthesia, but also from the trauma associated with a greater length of time before the closing of the incision or wound is accomplished. In some operations, there may also be damage from the length of time the patient spends on a heart-lung machine.

An additional disadvantage to current methods of using flexible suture material is the cost. Because of the length of time involved in stitching, doctors do not use the same suturing needle for very many stitches. It is faster to pick up a new one than to use all of the thread on the first. Therefore, they are disposed of very rapidly, often after only one suture. Wholesale costs of these products range from $5 to $20 and more per suture. Hundreds may be used in a single operation, putting the cost to the surgeon or hospital at thousands of dollars for a complicated operation requiring many sutures.

A great deal of skill is required to properly align critical stitches made at any level by hand. The unsteadiness of the hand is magnified by the nature of the activity, which requires repeated careful placement of the needle's tip, often while held at the end of a long clamp. Hand stitching may require a complicated circular piercing motion involving rolling the wrist and articulating the fingers. The above mentioned release and regrasping of the needle followed by a long pull of the needle away from the site of the suture necessitates the same precise targeting by the operator as the original entry. Either improper alignment and/or irregular spacing places the success of any suturing procedure, and possibly the entire operation, at risk.

However, the use of flexible sutures does provide advantages that obviously outweigh the above-described slow, inaccurate and consequently life-threatening process. Some of those advantages include the mechanical constraint of a flexible filament permits a small but important degree of variation in the alignment of separate pieces of tissue during the post-operative period. This probably induces lower levels of inflammation in the healing tissue. Such small variations also act as miniature shock absorbers allowing stresses applied to said joint to be relieved by means other than deformation of the tissue alone. Rigid means of fastening pass all such adjustment for potentially traumatic mechanical stress on to the tissue. Flexible filaments are available in absorbable and non-absorbable varieties, which gives the surgeon the choice of guaranteed release from constraint after the healing process is completed.

Stapling as a method of joining tissue arose in response to the need for a more rapid means of securing separate pieces of tissue. However, staples cannot be used for many surgical applications because of their inflexibility and inability for the body to absorb them. Staples, unlike filament, cannot be absorbed by the body following the surgical procedure and during or after the healing process. For instance, in coronary bypass surgery, surgeons are reluctant to use staples on the heart because of the constant muscular activity of that organ and the delicate nature of injured and weakened tissue. Staples may be also unsuitable for closure of tissue in operations involving the desire for minimal scaring, such as plastic surgery or intramuscular suturing procedures where scaring could adversely affect muscular performance.

### SUMMARY

In response to these and other problems, in one embodiment, there is disclosed a method of use and a suturing device comprising of: an arcuate needle, a plurality of one way gate mechanisms radially spaced around a portion of the arcuate needle, wherein each one way gate mechanism engages the needle in one direction, and a driving means for driving the plurality of one way gate mechanism around a portion of the needle such that the needle is incremented along a rotational direction.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1. is a perspective view illustrating one embodiment of a suturing device which incorporates one or more aspects of the present invention.

FIG. 2 is a perspective view illustrating one aspect of a needle incrementing mechanism which may be incorporated into a suturing device such as illustrated in FIG. 1.

FIG. 3 is an exploded front view showing some of the primary structural elements of the needle incrementing mechanism illustrated in FIG. 2.

FIG. 3a is an exploded back view showing some of the primary structural elements of the needle incrementing mechanism illustrated in FIG. 2.

FIG. 4 is a detailed plan view of the needle incrementing mechanism illustrated in FIG. 2 with the outer cases removed.

FIG. 4a is a perspective side view of needle incrementing mechanism illustrated in FIG. 4.

FIG. 4b is a perspective rear view of the needle incrementing mechanism illustrated in FIG. 4.

FIG. 5 is a detail perspective view of one aspect of a blade which may be incorporated into the needle incrementing mechanism illustrated in FIG. 4.

FIG. 6 is a detailed perspective view of one aspect of a needle which may be incorporated into the needle incrementing mechanism illustrated in FIGS. 2 and 4.

FIG. 7 is full perspective top view of a reversing element which could be implemented in some aspects of the present invention.

FIG. 7a is a detail partial perspective end view of the reversing element illustrated in FIG. 7.

FIG. 7b is a detail partial perspective view of the reversing element illustrated in FIG. 7.

FIG. 7c is a full perspective bottom view of the reversing element illustrated in FIG. 7.

FIG. 7d is a detailed partial perspective bottom view of the reversing element illustrated in FIG. 7.

FIG. 8 is an enlarged detail of a blade and cavity of the mechanism illustrated in FIG. 4.

FIG. 8a is the enlarged detail of FIG. 8 with a needle removed for clarity.

FIG. 8b is the enlarged detail of FIG. 8a showing the blade a different position than illustrated in FIG. 8a.

FIG. 9 is one embodiment of a reciprocating housing which may be used in one embodiment of the present invention.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

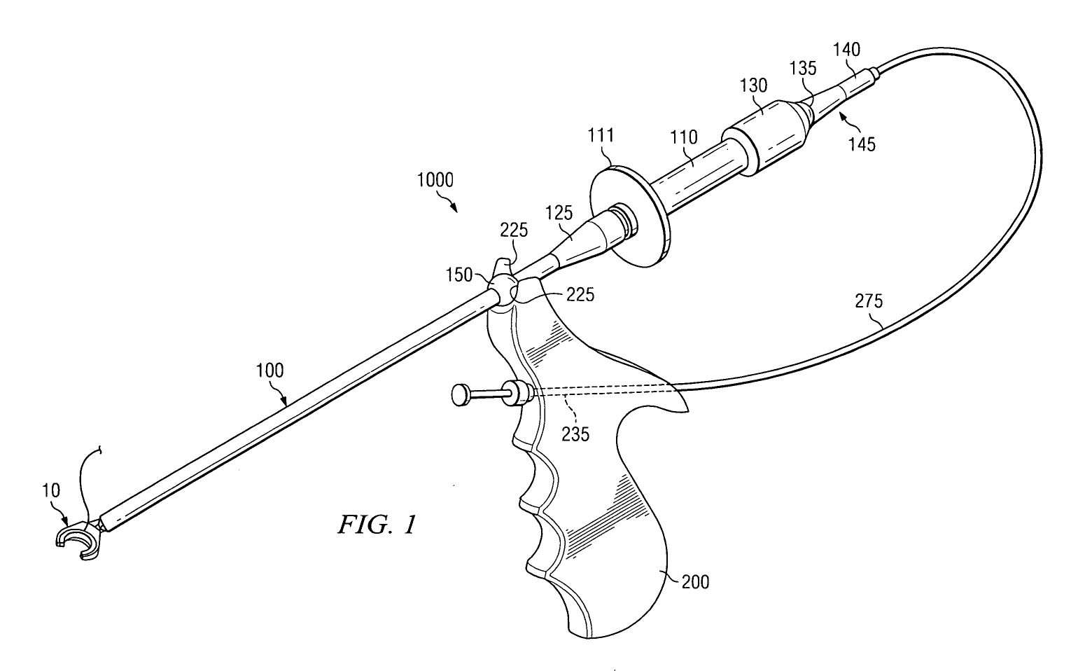

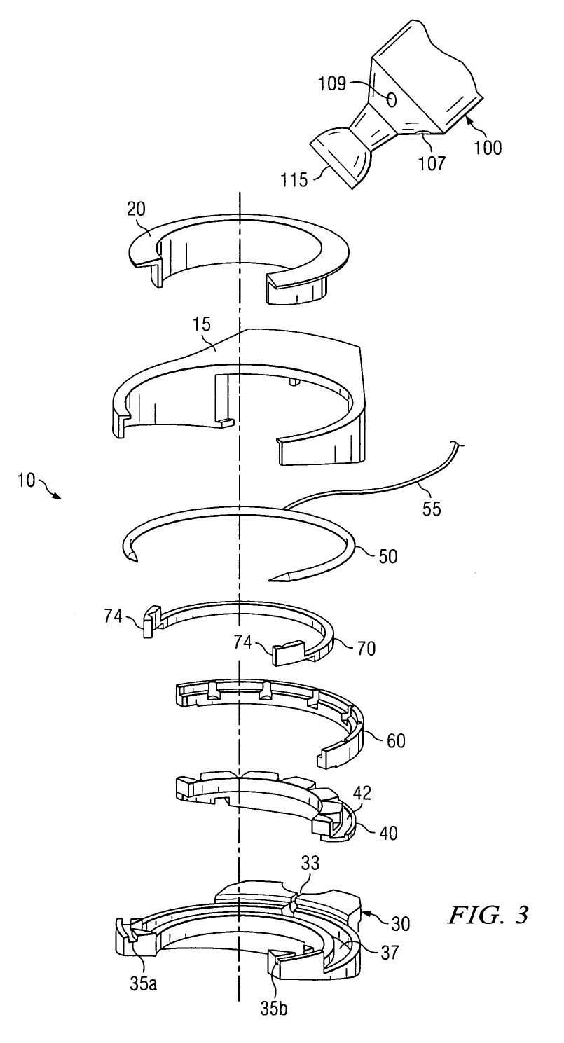

Turning now to FIG. 1, there is presented one embodiment of a suturing and knot-tying device 1000. In the illustrated embodiment, a crescent shaped needle incrementing mechanism 10 may be coupled to the suturing and knot-tying device 1000 as indicated in FIG. 1. The needle incrementing mechanism 10 may also be couple to other embodiments of suturing and knot-tying devices, such as illustrated in FIG. 3 of the inventor's prior application Ser. No. 10/263902, entitled “Cycling Suturing and Knot-Tying Device,” filed on Oct. 2, 2002, which has been previously incorporated by reference in its entirety into this application.

In the illustrated embodiment, the needle incrementing mechanism 10 may be coupled to a distal end of an extension tube 100 such that the needle incrementing mechanism 10 may rotate about the end of the extension tube. As illustrated, the needle incrementing mechanism 10 is articulated slightly up and to the operator's left. A handle 200 is shown in an upright position coupled to a rotation ball 150 which may be supported by a partial socket 225 of the handle 200. A distal end of a transition tube 125 may be coupled to the proximal end of the extension tube 100. Similarly, a distal end of a transmission tube 110 may be coupled to the proximal end of the transition tube 125. In the illustrative embodiment, a slidable articulation socket 130 may be coupled to the proximal end of the transmission tube 110. In some embodiments, the slidable articulation socket 130 may contain an articulation lever joint ball 135. A level assembly 145 may be coupled to the proximal side of the articulation lever joint ball 135. In some embodiments, an articulation handle 140 may be coupled to the level assembly 145.

In some embodiments, a flexible cable plunger/actuator 275 may be mounted in a bore 235 of the handle 200. The flexible cable 275 enters the transmission tube 110 through the level assembly 145. In some embodiments, the plunger/actuator 275 may act to drive a cable circuit which drives the needle incrementing mechanism 10. In other embodiments, the plunger/actuator may be coupled to the articulation system to also position the needle incrementing mechanism 10.

It will be appreciated that the transmission tube 110 can be articulated to the left or right with respect to the handle 200 to precisely position the needle incrementing mechanism 10 in a desired location with respect to tissue to be sutured. Accordingly, the extension tube 100 and needle incrementing mechanism 10 can be articulated to any selected angle from a far left position to a far right position (not illustrated). Similarly, the extension tube 125 can be pitched downwardly and upwardly throughout a wide angle of pitch by pivoting the rotation ball 150 on the extension tube 110 with respect to the handle 200 thus further facilitating precise positioning of the needle incrementing mechanism 10 in a desired position in close quarters during a suturing operation.

In some embodiments, a second handle (not shown), having a handle trigger, may be attached to the handle by means of a flexible connection to control a thread incrementing accessory (not illustrated) mounted on the needle incrementing mechanism 10.

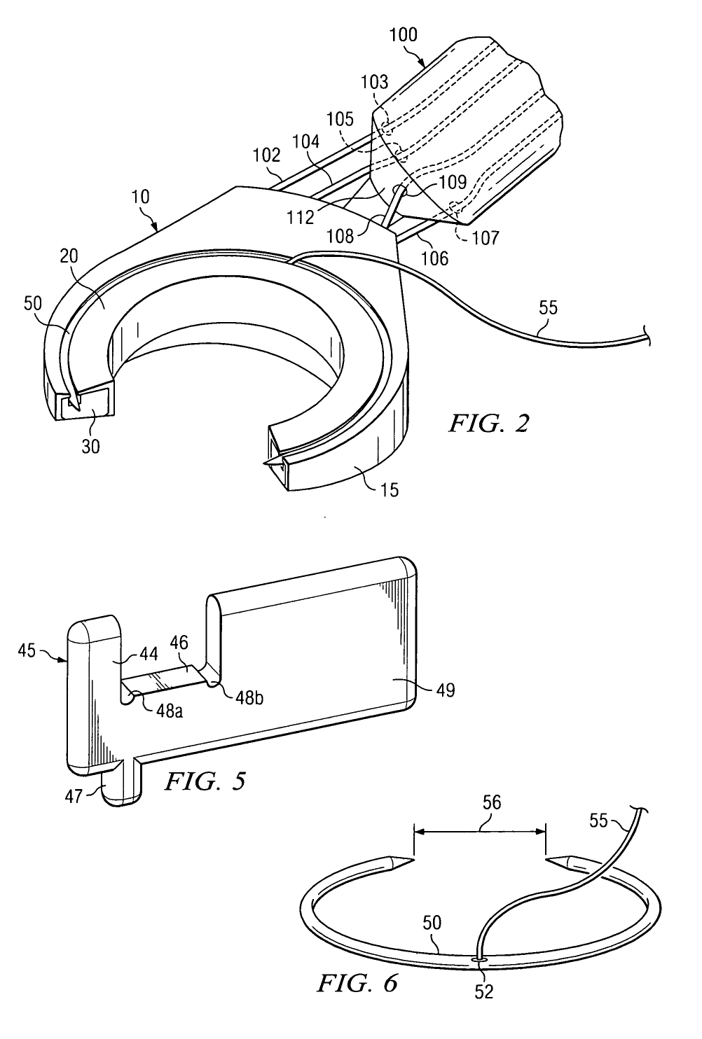

FIG. 2. illustrates a detailed enlarged view of one embodiment of the needle incrementing mechanism 10. In some embodiments, the needle incrementing mechanism 10 may be coupled a articulation ball 115 (not shown in FIG. 2). The articulation ball 115 may be located on the distal end of the extension tube 100. In some embodiments there may be one or more transition cones 112 which provide for a transition between the fixed articulation ball 115 and the extension tube 100. In several embodiments, the articulation ball 115 may be fixed and adapted to be seated in a socket cavity 32 (not shown in FIG. 2) provided in a back surface of the needle incrementing mechanism 10. Such a connection facilitates universal articulation of the needle incrementing mechanism 10 with respect to the extension tube 110.

Controlled universal articulation of the needle incrementing mechanism 10 on the articulation ball 115 may be facilitated by four angle articulation cables 102, 104, 106 and 108, respectively, typically extending from within the transition cone 112, through corresponding cable holes 103,105,107 and 109, respectively.

The extending end of each of the articulation cables 102,104, 106 and 108 may be attached by any suitable method to corresponding base corners on a back face of the needle incrementing mechanism 10. The opposite ends of these cables may be coupled to the articulation lever joint ball 135, such that manipulation of the articulation handle 140 (FIG. 1) moves the articulation lever joint ball 135 so that tension may be applied to the corresponding one of the articulation cables 102, 104, 106 and 108, to manipulate and articulate the needle incrementing mechanism 10 into a desired position with respect to the tissue.

In some embodiments of the invention, the articulation cables 102, 104, 106 and 108 extend from the lever assembly 145, through the transmission tube 110, the transition tube 125, the extension tube 100 and through the corresponding transition guide cone cable inlet holes 103, 105, 107 and 109, respectively, in the transition guide. cone 112.

A thread 55 is also illustrated in FIG. 2. In the illustrated embodiment, the thread 55 may be coupled to a center of a crescent shaped needle 50. In some embodiments, the needle 50 has two ends, each pointed and adapted to enter into tissue. The needle 50 may be housed in a fixed way 30. An outer case 15 and an inner case 20 may surround and house the fixed way 30.

Referring now to FIGS. 3 and 3A, there is illustrated exploded views of one embodiment of the needle incrementing mechanism 10 and the distal end of the extension tube 100. In some embodiments, a crescent shaped fixed way 30 functions as a base for the needle incrementing mechanism 10 and as a guide for some of the moving components. A continuous slot or drive guide channel 37 may be defined in the upper face of the fixed way 30. A driver 40 may be adapted to slidably engage the fixed way 30.

In some embodiments, the driver 40 works with a reverser 60 to position a plurality of one-way engaging devices, such as a plurality of blades, radially about the needle 50. As will be explained in detail later, in some embodiments, the driver 40 may cause the plurality of blades to engage the needle 50. Consequently, as the driver moves, the needle 50 also moves.

The driver 40 may have a lower protrusion 40A (FIG. 3A) adapted to slidably mate the drive guide channel 37. The protrusion 40A may be circumferentially shorter than the channel 37 so that the protrusion 40A can slide within the channel 37 when driven by a driving means such as drive cables coupled to each end of the driver 40. In some embodiments, at each end of the fixed way 30, there may be needle guide channels 35a and 35b for guiding the needle 50 into and out of the needle incrementing mechanism 10.

In some embodiments, a continuous cable circuit (not shown) may be attached to either end of the driver 40 to move the driver within the fixed way 30. The cable circuit may loop through a drive cable channel 33 in the needle incrementing mechanism 10 and also through a passage in the articulation ball 115 on through the extension tube 100 to the transmission tube 110, where it may be tensioned as described below.

In other embodiments, the driver 40 may be coupled to a drive cable circuit such as described in the inventor's prior application Ser. No. 10/263902, entitled “Cycling Suturing and Knot-Tying Device,” filed on Oct. 2, 2002, which has been previously incorporated by reference in its entirety into this Application. In such an embodiment, the opposite ends of the drive cables are typically connected to a cable driving apparatus to facilitate selective incrementation of the reciprocal driver 40 in both the clockwise and counterclockwise directions and incrementally drive the needle 50 around the fixed way 30 in a circular direction.

The reverser 60 may be slidably contained within a groove 42 on the outside upper face of driver 40. As will be explained later, in some embodiments, the reverser 60 may be coupled with the plurality of the one-way engaging devices (e.g., the plurality of blades) and may work with driver 40 to change the direction of the one-way engaging devices.

In some embodiments, the fixed way 30 may also be coupled to one way engaging devices that are also adapted to engage the needle 50. In some embodiments, a fixed way reverser 70 may be coupled to the engage devices and adapted to work with the fixed way 30 to change the direction of the one-way engaging devices controlled by the fixed way. The fixed way reverser 70 may be contained by the inside radius of the reverser 60. The reverser 60 may be longer than the driver 40 and when set in a direction, one end of the reverser 60 is even with the end of the driver.

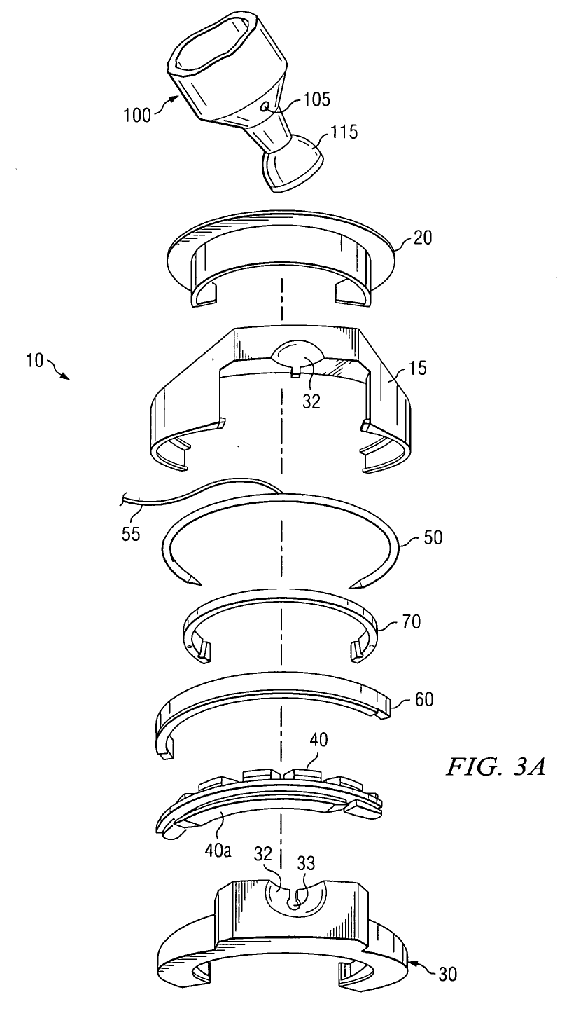

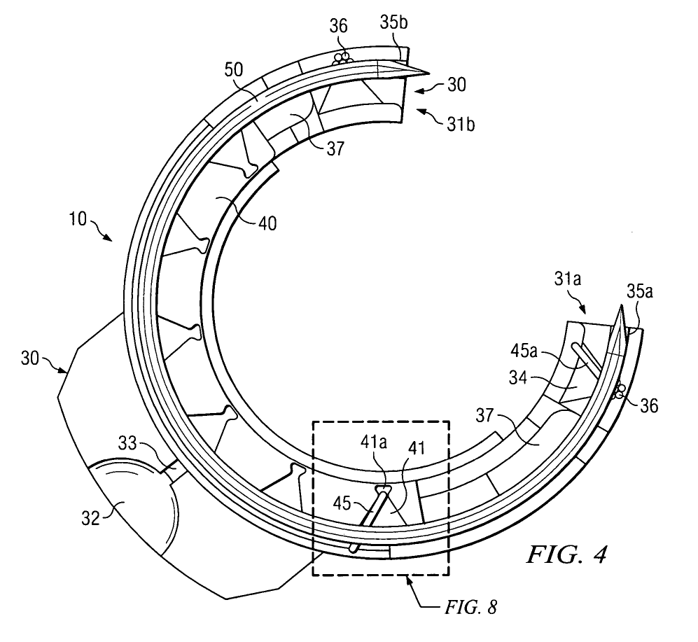

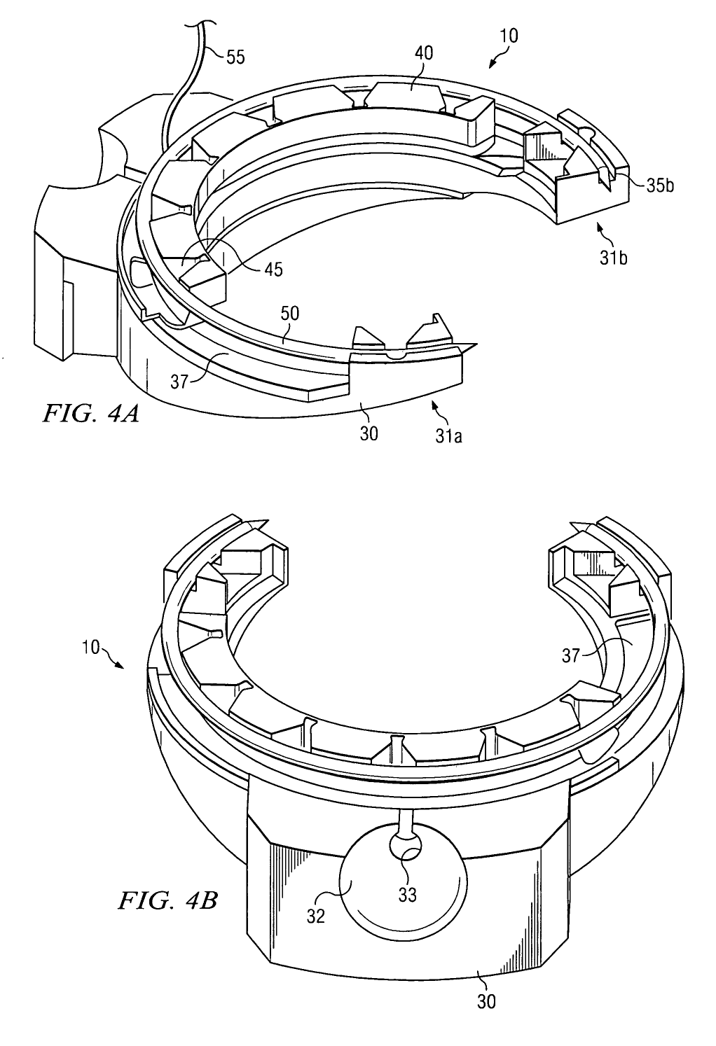

The driver 40, the reverser 60, the fixed way reverser 70 and the associated engaging devices are positioned for accommodating and stabilizing the curved needle 50 which is provided with a length of thread 55. As further illustrated in FIG. 4, the curved needle 50 extends through a groove or needle drive channel 35a and 35b provided in the ends 31a and 31b of the fixed way 30.

Assembly of the respective components of the illustrative embodiment of the needle incrementing mechanism 10 is illustrated in FIGS. 3 and 3A. The fixed way 30, the driver 40, the reverser 60, and the fixed way reverser 70 may be all stacked and contained in a crescent shaped outer case 15 and an arcuate shaped inner case 20. The outer case 15 and the inner case 20 maintain the components in place. As can be seen in FIG. 3B the combination of the fixed way 30 and the outer case 15 form a socket 32 which may be adapted to mate with the articulation ball 115 of the extension tube 100.

Turning now to FIG. 4, there is illustrated a plan view of one aspect of the needle incrementing mechanism 10 with outer case 15 and inner case 20 removed to show the driver 40 installed in the fixed way 30. In some embodiments, there are a plurality of triangular-shaped cavities 41 defined in the outer circumferential surface of the driver 40. Each of the cavities 41 narrow to an apex, then expands to form a narrow relief sub-cavity 41A. Each cavity 41 and each sub-cavity 41 may hold a portion of a blade 45. However, for clarity, only one blade 45 is shown in FIG. 4.

In some embodiments, proximal to ends of the fixed way 30, there may be a triangular cavity 34 defined in the interior circumferential surface of the fixed way 30. Each triangular cavity 34 may narrow to an apex, then expands to form a spring support cavity 36. Each cavity 34 and sub-cavity 36 may hold a portion of a blade 45a. For clarity only one blade 45a is shown in FIG. 4. As will be explained below, a slot may be defined in the blades 45a to engage and support the needle 50.

A previously discussed, in some embodiments, the fixed way 30 may have a socket 32 defined in the rear surface and the drive cable channel 33. Also visible from this view is the needle guide channels 35a and 35b, and the fixed way driver guide channel 37.

Turning now to FIG. 4A, there is illustrated a perspective of one embodiment of a needle incrementing mechanism 10 with elements removed to indicate the relative range of movement for the driver 40 within the fixed way driver guide channel 37. As illustrated, in this embodiment, the driver 40 may be slidably disposed within the driver guide channel and may move in an arc between the fixed way ends 31a and 31b. The needle 50 is also illustrated positioned within the needle drive channels 35a and 35b and in a slot defined with a blade 45.

FIG. 4B is another perspective of needle incrementing mechanism 10 viewed from the rear showing some of the components. Namely, the partial socket 32 may be defined in the rear surface of the fixed way 30. Also shown is the drive cable channel 33.

Turning now to FIG. 5, there is illustrated a perspective view of blade 45 or 45a. In some embodiments, the top surface of the blade 45a defines a notch 46 for receiving the needle 50 (not shown in FIG. 5). In some embodiments, there are debris relief channels 48a and 48b which allow tissue debris to be removed from the surfaces of the notch in contact with the needle 50. As explained previously, the blade 45 may be adapted to fit within the cavities 41A and 41 (or cavities 34 or 36 of the fixed way). An end 49 may be adapted to fit within the cavity 41A and the opposite end 44 may be adapted to couple with a reversing element such as the reverser 60. As will be explained below the blade 45 may be adapted to pivot with the cavities about a pivot post 47.

FIG. 6 is a perspective illustration of one embodiment of the needle 50, illustrating a central attachment of the thread 55 through a crimp eye 52. Also shown is a needle gap 56.

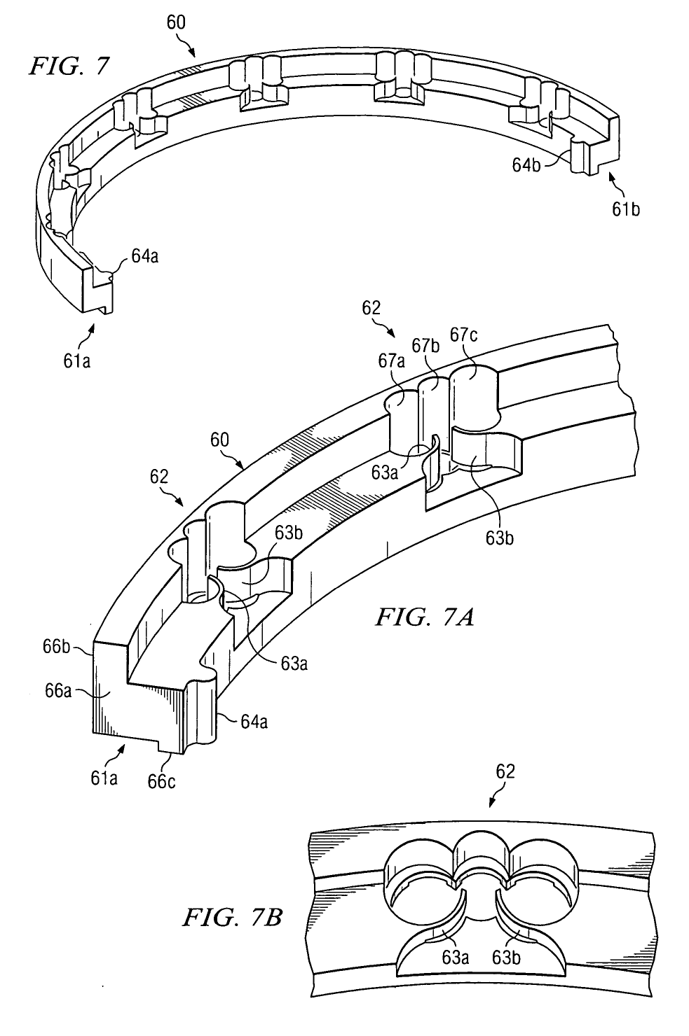

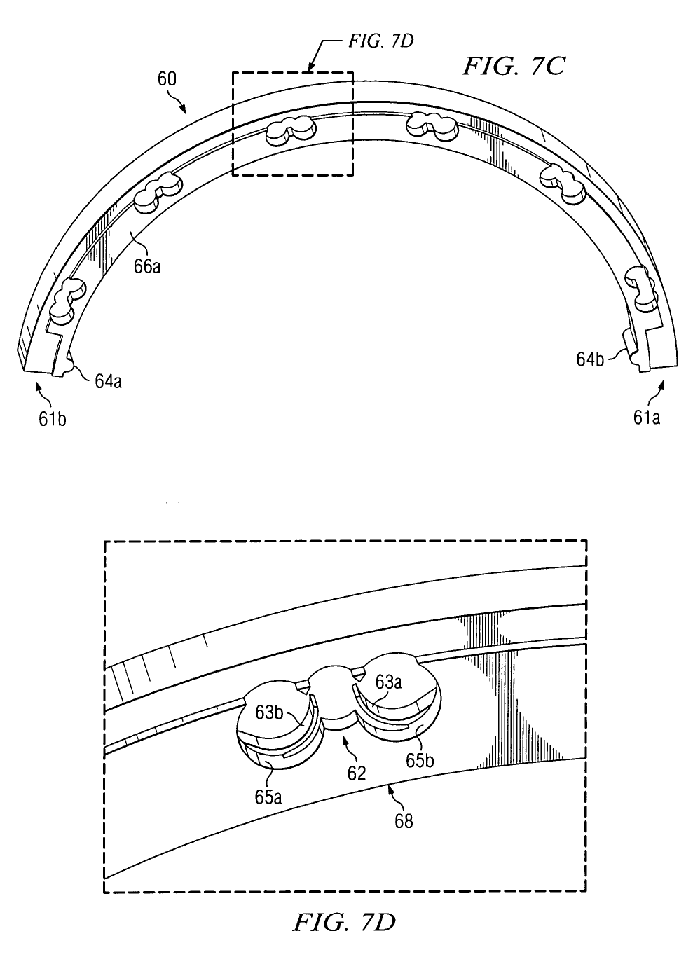

Turning now to FIG. 7, there is illustrated a top perspective view of the arcuate reversing element or reverser 60. Two bosses 64a and 64b are positioned on the interior surface of the reverser proximal to each end 61a and 61b. As will be explained below, the bosses 64a and 64b are adapted to engage and mate with a corresponding detent defined in the exterior radial surface of the driver 40. A plurality of cavities or indentations are defined on the interior radial surfaces of the reverser 60 which correspond to the cavities 41 of the driver 40b.

A partial perspective detail of one end 61a of the reverser is illustrated in FIG. 7A. In the illustrative embodiment, in cross-section, the reverser 60 may have a square base section 66a. In some embodiments, there may be an exterior vertical upper flange section 66b projecting from the base section 66a. In some embodiments, there may be an interior vertical lower flange section 66c projecting down from the base section 66a. Proximal to the end 61a is a spring support cavity 62. In contrast to the cavities 41 discussed with reference to the driver 40, support cavity 62 may be generally curved. Protuding into the cavity 62 are integral spring leaves 63a and 63b. In some embodiments, corresponding to the support cavity 62 are three vertical overlapping bores 67a, 67b and 67c which form detents on the interior face of the vertical flange 66b.

A boss 64a (one of two) may be located proximal to the end 61a. The boss 64a fits into detent 65 (one of two neither of which is shown) on driver 40. As will be explained below, The boss 64a holds the reverser 60 in its direction setting position at the extreme end of its range of motion relative to driver 40. A partial perspective detail view of the support cavity 62 is illustrated in FIG. 7B. In this view, the protrusion of the springs 63a and 63b are clearly illustrated protruding into the support cavity 62.

Turning now to FIG. 7C., there is illustrated a full perspective view of the reverser 60 shown from beneath. As can be seen from this angle, in this embodiment, the lower flange 66a extends longitudinally along the entire length of the curved reverser 60. However, the lower flange 66a narrows in width as the lower flange approaches the ends 61 a and 61 b of the reverser 60. FIG. 7D. is an enlarged detailed perspective view of one embodiment showing the spring support cavity 62 and leaves 63a and 63b from below. In this embodiment, the leaves 63a and 63b may be disconnected from the surrounding structure 68 by means of offset lower cavities 65a and 65b.

Operation

Referring now to FIGS. 1 through 7D, the manner of using one embodiment of the present invention will now be described. The leverage locking reversible cyclic suturing and knot-tying device 1000 automatically sutures surgical incisions, wounds or other material that needs to be joined together by stitching. To propel the needle 50 and attached thread 55 through any substance placed within the needle and supporting structure's gaps and thereby accomplish stitching, the driver 40 may mechanically lock or engage the needle 50 by means of at least one one-way locking mechanism and increments it forward or in reverse until it has passed completely through the material, trailing the thread behind it to make a stitch.

In some of the embodiments, the one-way locking mechanism may be a plurality of blades 45. Each blade 45 as a leverage locking device causes it to grip with increasing force when the needle 50 is moved against its leverage and open its grip by pivoting slightly when the needle is moved with the leverage (opposite directions). To aid the locking process, it may be helpful to use a spring or a springy quality in the blade 45 itself to maintain locking contact with the needle 50. In some embodiments, the blade 45 may then be pushed into contact with the needle 50 and held there with gentle pressure of a spring or similar device (e.g., the spring leaves 63a and 63b). The blades 45, therefore, may act as one-way gates which may engage the needle 50 without the need of special gear teeth, notches or other features in the needle which may weaken the needle or raise its resistance to passing through tissue.

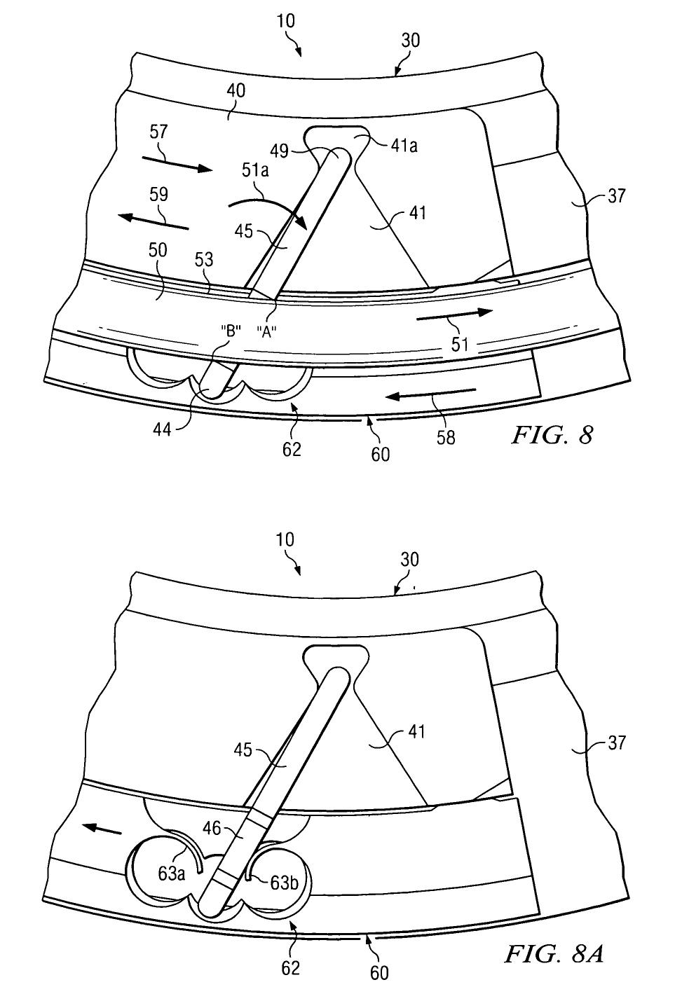

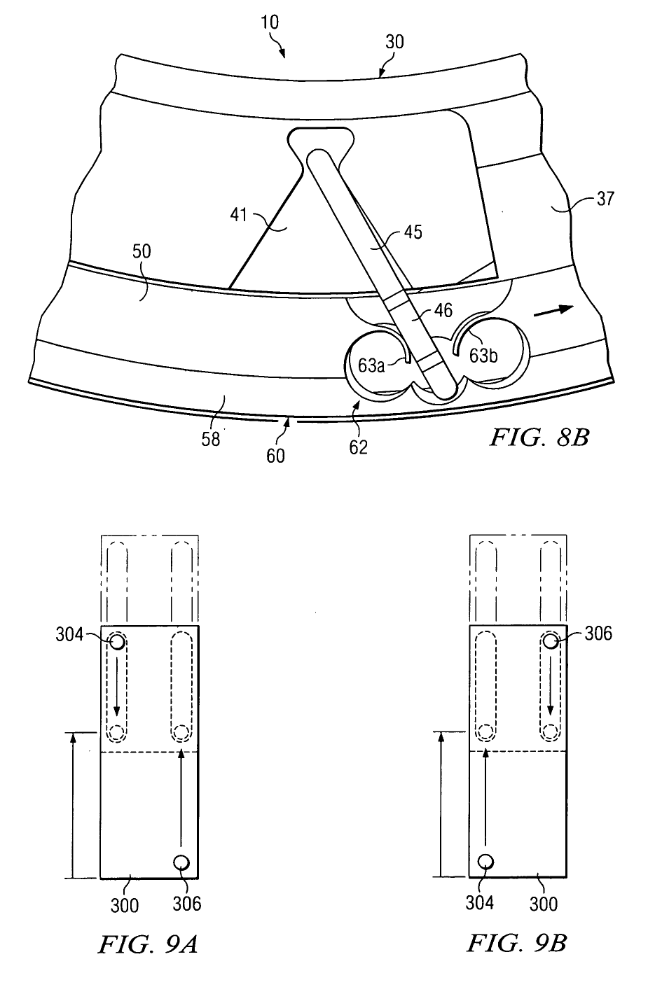

Turning now to FIG. 8, there is illustrated a detail of one embodiment showing the blade 45 engaging the needle 50. FIG. 8. is an enlarged detail view of the embodiment illustrated in FIG. 4 showing the blade 45 installed in a cavity 41 of the driver 40. In this illustrative example, the driver 40 may move relative to the fixed way 30 along the driver guide channel 37. Also shown in this detail (but not shown in general view of FIG. 4) is the reverser 60. The end 44 of the blade 45 may be positioned in the reverser/driver spring support cavity 62 of the reverser to support the end 44 of blade 45. The opposite end 49 of the blade 45 may be positioned in the pivot relief sub-cavity 41A as illustrated. Thus, allowing the blade 45 to pivot about its pivot post 47 (not shown) from a first position (as illustrated in FIG. 8) to a second position where the blade does not engage the needle 50.

As can be seen, the driver 40 rotates the blade 45 about its pivot post 47 (not shown) in a clockwise direction indicated by the clockwise blade rotation arrow 51a causing the blade 45 to engage the needle 50 at diagonally or diametrically-opposed edges A and B. This engagement is effected along with the rotation of the blade 45 by rotation of the driver 40 in the direction of the rotation arrow 57. Consequently, the needle 50 is now in position for counterclockwise rotation in concert with the driver 40 in the direction of the counterclockwise housing/driver/needle rotation arrow 51.

Thus, the direction of movement of needle 50 may be shown by arrow 51. The direction of needle incrementing movement of driver 40 may be shown by arrow 57. The direction of driver's repositioning at original starting point for further reciprocation incrementation may be shown by arrow 59. The direction of setting movement of reverser 60 may be shown by arrow 58.

Turning now to FIG. 8A, there is an enlarged perspective detail of some structural components which are visible in FIG. 8. However, in this illustration, the needle 50 is removed to show integral spring leaves 63a and 63b of the reverser 60. The leaf spring 63b of reverser/driver spring support cavity 62 is shown applying spring tension to the blade 45, which is angled to left of cavity 41. This tension assures contact of blade notch 46 with needle 50 (not shown). Thus, force by the driver 45 may be placed on the long end 49 of the blade 45, causing it to act as a lever to tighten the notch on the needle and move it in that direction with the leverage.

Turning now to FIG. 8B, there is illustrated an enlarged perspective of detail of structure visible in FIG. 8 and FIG. 8A with needle 50 removed to show integral spring leaves 63. In this illustration, the leaf 63a has traversed to the right side of reverser/driver spring support cavity 62 and is shown applying spring tension to blade 45, which is angled to the right of cavity 41. In this position of the blade lever acts upon needle 50 to increment it in the direction opposite to that shown in FIG. 8A.

In some embodiments, the direction of these one-way gates or blades 45 can be changed by flipping the blade 45 into the opposite locking angle and repeating all the common driving motions which increment the needle in either position. The change of angel may be accomplished by moving one end of the blade 45 while holding the other end immobile.

In some embodiments, setting movement of the reverser 60 establishes its relative position with regard to the driver 40. This position determines direction of needle incrementation. Once the reverser position is set relative to the driver 40, the two parts may move together in that relative position during the incrementation of needle 50.

As previously discussed, in some embodiments, a continuous cable circuit attached to either end of the driver 40 loops proximally through central hole in mechanism articulation ball 115 through extension tube 100 to transmission tube 110, where it may be tensioned with an idler pulley. The cable circuit may be provided with three crimped-on stops. Two of the stops are attached opposite each other on the left and right lengths of cable within transmission tube. They function as the drive stops, which actually apply incremental force through the driver to the needle in order to advance it.

In FIG. 9, there is illustrated a housing 300 which, in some embodiments, may reciprocate within the transmission tube 110 transferring force applied by a manually driven plunger (or electrical, pneumatic or other mechanical means to drive cable circuit through either crimp stops 304, 306.

The third stop may be spaced a distance away from the driving stops on one side of the circuit, and may be connected to sliding collar 111 (FIG. 1). The collar 111 may function as a selection switch with two positions, reverse and forward.

When the collar 111 is moved from one position to the other, a cable (not shown) of the actuation circuit within the transmission tube 110 pulls the driver 40 beyond its incremental range into a small range of motion set aside for changing the positions of the two reverser components. As previously discussed, the reverser 60 may be slidably contained within a groove on the outside of driver 40, while fixed way reverser 70 may be contained by the inside radius of the driver. The reverser 60 may be longer than driver 40 and when set in a direction, one end of the reverser 60 is even with the end of the driver. The other end of the reverser 60 protrudes a short distance beyond the end of the driver 40. When the driver 40 reciprocates in this position, it stops short of touching with this protruding end, the end of the range of motion within the fixed way 30.

When collar or reversing switch 111 causes the driver 40 to move past this stopping point, this protruding end comes in contact with the end of the range of motion and stops, while the driver 40 continues to move until it also reaches the end of the range of motion.

At this point, one of the bosses 64a or 64b has been forced out of its corresponding detent 65 on the outside corresponding end of driver 40, and caused to protrude beyond the end of the driver. At the same time, the opposite end of the reverser 60 which was formerly protruding is now brought back even with the end of the driver, so that its boss may be then captured by the detent on the corresponding outside end of the driver 40.

As this is taking place, a reversal of direction may occur for the two blades 45a positioned in the fixed way 30. The fixed way reverser 70 (longer than either the driver 40 or its reverser 60) extends almost the full distance of the fixed way. It stops short (leaving just enough space) for it to provide a similar function by being shifted between two positions and moving the two fixed way blades by doing so. Because of the need to reverse both sets of blades (e.g., those of the driver 40 and those of the fixed way 30, it may be necessary to engage the opposite ends of the fixed way blades in order to reposition them. This permits the arc-shaped reverser, which may extend around the entire length of the fixed way so that it can affect both blades simultaneously to be placed on the inside of the fixed way's arced structure. The fixed way reverser 70 may by designed so that a portion of the blade changing structure protrudes into the same space that the end of reverser 60 occupies when it protrudes beyond the end of the driver.

When the driver 40 is forced into this space (in order to change the direction of its own blades), it may also encounter the structure of the fixed way reverser 70 by contacting it with its own end which may force it to change position at the same time as the structure of the fixed way is forcing reverser 60 to change position. They are however, moving in opposite directions with respect to each other, and the blades of both are be angled in the same direction with respect to the needle 50. The consequence of this is that the fixed way blades are moved by their inner lever ends 49 so the cavities of the fixed way that control the blades are laid out in the opposite direction. The spring connection with the spring mounting portion of the blade 45 is stationary in the fixed way. Similar bosses 74 on the ends of reverser 70 mate with detents 75 on fixed way 30 to hold this angle determining component reverser 70 in either on of its positional options.

Thus, disclosed aspect provides an automatic means of passing a flexible thread 50 through a penetrable, usually deformable material, and drawing together the ends of the thread 50 and joining them. This process may be used to contain two or more pieces of material in juxtaposition to one another, or one piece of material in a certain condition of deformation. The above activity may be accomplished by the motorized application of power initiated and controlled by remote, discrete switching means, which affect the position of the device as little as possible. The reason for this limitation of extraneous movement is to minimize the difficulty of manually placing the entry and exit of said thread. This permits the operator to move the device directly to the site of each new penetration or external tying, looping or lacing procedure, in a very short time and without the necessity of other manual sub-procedural steps which greatly affect the precision and speed of placement.

Unlike existing methods of passing a thread through material, this, device does not require the pulling away of the needle from the area of activity in order to advance the thread through the penetration site. This advancement of the thread permits the first site of penetration and exit to be placed at the end of the thread, and each successive site to be placed sequentially closer to the beginning point of the thread, which is attached to or retained by the needle. This device passes the needle all the way through the material and picks it up on the other side, while maintaining a precise distance between entry and exit, which can be adjusted or left the same from stitch to stitch. The thread is drawn through behind the needle so as to leave inside the area of penetration a portion of the thread closer to its terminus, and to prepare for the next entry or looping, tying or lacing procedure. The thread can be optionally gripped or not gripped on both sides of the penetration. This depends on whether the next procedure demands the present one to be tied off or if one or more additional entries, loops, etc., will be performed as part of a longer procedure, before termination by tying or fusing.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. When the word “means” is recited in a claim element, Applicant intends for the claim element to fall under 35 U.S.C. 112, paragraph 6. Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word means are not intended to fall under 35 U.S.C. 112, paragraph 6. Signals are typically electronic signals, but may be optical signals such as can be carried over a fiber optic line.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

For instance, in some embodiments, there may be an suturing device comprising: an arcuate needle having an interior face and an exterior face, a plurality of blades radially spaced around a portion of the arcuate needle, wherein each blade is adapted to pivot from a first position to a second position and in the first position each blade is adapted to engage the interior face and the exterior face of the needle and in the second position the blade disengages the interior face and exterior face of the needle, a plurality of springs adapted to maintain the position of each blade in the plurality of blades against the needle when each blade is in the first position, a driving means for driving the plurality of blades radially around a portion of the needle such that the needle is incremented along a first rotational direction, and a fixed way means for providing a path for the driving means.

In other embodiments, there may be a suturing device comprising: a curved needle, a plurality of blades radially spaced around a portion of the curved needle, wherein each blade has a slot having interior faces for engaging a sub-portion of the needle when the blade is in a first position and wherein the interior faces of the slot do not engage the needle when the blade is in a second position, a plurality of springs adapted to maintain the position of each blade in the plurality of blades against the needle when each blade is in the first position, a drive means for driving the plurality of blades radially around the needle such that the needle is urged along in a first direction, and, a fixed way means for providing a path for the driving means.

In yet other embodiments, there may be a suturing device comprising: a curved needle for advancing a thread along a path, a first plurality of one-way engaging devices for engaging the curved needle, an arcuate reversing means concentrically positioned about the curved needle for changing a direction of the one-way engaging devices wherein the arcuate reversing means is coupled to the plurality of engaging devices, an arcuate driving means concentrically positioned about the curved needle wherein the driving means moves along a path and is adapted to pivot the plurality of engaging devices causing the plurality of engaging devices to engage the needle, and a fixed way means for providing the path.

In such embodiments, the one-way engaging devices are blades having a slot wherein the slot is adapted to engage the needle. Furthermore, the fixed way means may have a second plurality of pivoting one-way engaging devices for engaging the curved needle, and a second reversing means adapted to change a direction of the second plurality of one-way engaging devices. There may also be a direction setting means for setting the rotational direction of the movement of the needle. The direction setting means may be an arcuate structure coupled to the plurality of blades. The fixed way means my be adapted to support and aligning the driving means, wherein the driver means is slidingly coupled to the fixed way means.

Some embodiments may also comprise a drive circuit means for driving the driving means. The drive circuit means comprise at least one tensioning means coupled to the driver means for pulling on the driver. Such an embodiment could also comprise at least one pulley means coupled to the tensioning means for keeping the tensioning means taught, and at least one advancing means coupled to the tensioning means for selectively pulling the tensioning means. The embodiments could comprise an articulation means for positioning the arcuate frame. The articulation means could be selected from the group consisting of a ball and socket joint, a segmented neck, or a universal joint hinge. The suturing device could further comprise an articulation control means for adjusting the articulation means. The articulation control means further comprises: a tensioning means coupled to the arcuate frame, and a ball means coupled to the tensioning means such that when the ball means is rotated the tensioning means pulls on the arcuate frame to adjust the position of the arcuate fame.

The embodiments could further comprise a thread pulling means for pulling the thread during suturing. Some of the embodiments could further comprise a tubular means for housing the drive circuit means. Other embodiments could also comprise a handling means coupled to the tubular means for positioning the suturing device.

Other alternative embodiments could comprise a method of suturing comprising: providing a blade with a slot, wherein interior surfaces of the slot are adapted to engage a curved needle, pivoting a blade so that engagement between the interior surfaces of the slot and the needle occurs, maintaining the engagement of the blade against the needle with a spring-like force, driving the bade in a first direction such that the needle is also driven in a first direction, advancing a first end of the needle out of a housing, accepting the first end of the needle into the housing, coupling a thread to the needle such that the thread is pulled behind the needle as the needle moves out of the housing and back into the housing.

1. A suturing device comprising:
a curved needle for advancing a thread along a path,
a first plurality of one-way engaging devices adapted to selectively engage the curved needle,
an arcuate reversing housing concentrically positioned about the curved needle for changing a direction of the one-way engaging devices wherein the arcuate reversing housing also engages the plurality of engaging devices,
an arcuate driving means concentrically positioned within the curved needle wherein the driving means moves along a path and is adapted to pivot the plurality of engaging devices causing the plurality of engaging devices to engage the needle, and
a fixed way means for providing the path for the arcuate driving means.

2. The suturing device of claim 1 wherein the one-way engaging devices are blades having a slot wherein the slot is adapted to engage the needle.

3. The suturing device of claim 1 wherein the fixed way means has a second plurality of pivoting one-way engaging devices for engaging the curved needle, and a second reversing means adapted to change a direction of the second plurality of one-way engaging devices.

4. A method of surgically suturing tissue comprising:
providing a blade with a slot, wherein interior surfaces of the slot are adapted to engage a curved needle,
pivoting a blade so that engagement between the interior surfaces of the slot and the needle occurs,
maintaining the engagement of the blade against the needle with a spring-like force,
driving the bade in a first direction such that the needle is also driven in a first direction,
advancing a first end of the needle out of a housing,
accepting the first end of the needle into the housing, and
coupling a thread to the needle such that the thread is pulled behind the needle as the needle moves out of the housing and back into the housing.
